(19) United States
(12) Patent Application Publication
(10) Pub. No.: US 2022/0243251 A1
(43) Pub. Date: Aug. 4, 2022

(54) NUCLEOTIDES WITH ISOTOPES FOR DNA DATA STORAGE

(71) Applicant: Seagate Technology LLC, Fremont, CA (US)

(72) Inventor: Eric K. WADLEIGH, Shakopee, MN (US)

(21) Appl. No.: 17/166,838

(22) Filed: Feb. 3, 2021

(51) Int. Cl. C12Q 1/68; C07H 21/04

(52) U.S. Cl. CPC C12Q 1/68; C07B 2200/05; C07H 21/04

(57) ABSTRACT

Nucleotides are provided with at least one isotope. The isotope-modified nucleotides can be used for data storage, increasing the data density compared to only natural nucleotides. Described is a method of storing data on a DNA strand, the method comprising providing a DNA strand having at least one isotope-modified nucleotide comprising at least one isotope of carbon, nitrogen, oxygen or hydrogen, assigning a bit pattern to the at least one isotope-modified nucleotide that is different than a bit pattern assigned to a non-isotope-modified nucleotide. Data could be stored on any molecule that can be isotope-modified.

### BACKGROUND

Using DNA for storing data is an emerging technology.

Traditional biological DNA data storage is limited to four states; the state values are represented by the nucleotide present: (A) adenine, (C) cytosine, (G) guanine, or (T) thymine. A data storage bit is represented by one nucleotide on one half (single strand) of the DNA double strand; the other half of the DNA strand has the complementary nucleotide, which offers redundancy but not extra data capability.

### SUMMARY

This disclosure provides methodology that massively increases the amount of data that can be stored on DNA, with the theoretical storage limit exceeding 1 binary bit per atom. Particularly, this disclosure provides methodologies that utilize nucleotides, formed with at least one isotope of at least one of H, C, N or O. Other molecules, in addition to nucleotides, can be modified with one or more isotopes and similarly used. The isotope-modified nucleotides, and other molecules, can be used for data storage. The nucleotides, and thus the data they encode, can be read, e.g., by spectroscopy, such as Surface-Enhanced Raman Spectroscopy (SERS).

This disclosure provides, in one particular implementation, a method of storing data on a DNA strand. The method includes providing a DNA strand having at least one isotope-modified nucleotide comprising at least one isotope of carbon, nitrogen, oxygen or hydrogen, and assigning a bit pattern to the at least one isotope-modified nucleotide that is different than a bit pattern assigned to a non-isotope-modified nucleotide.

A similar method can be utilized for storing data on any molecule, crystal, or other material that can be isotope-modified in such a way that physical or logical order is maintained.

This disclosure provides, in another particular implementation, a DNA strand or an RNA strand encoding data, the DNA or RNA strand having at least one natural nucleotide having a first bit pattern assigned thereto, and at least one isotope-modified nucleotide comprising at least one isotope of one of carbon, nitrogen, oxygen or hydrogen, the isotope-modified nucleotide having a second bit pattern assigned thereto different than the first bit pattern.

This disclosure also provides, in another particular implementation, a system for data storage on a DNA strand. The system includes a plurality of isotope-modified nucleotides, each isotope-modified nucleotide comprising at least one isotope, and each isotope-modified nucleotide having a number of possible states. The number of possible states defined by (a^{Na})*(b^{Nb})*(c^{Nc})* . . . (z^{Nz}), where a, b, c . . . z is the number of isotopes available for a given atom, and Na, Nb, Nc . . . Nz is the number of atoms of type a, b, c, and z in the nucleotide.

A similar system can be used to store data on any molecule, crystal or other material that can be isotope-modified.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

The described technology is best understood from the following Detailed Description describing various implementations read in connection with the accompanying drawing, where:

FIG. 1A is the molecular structure of adenine (A); FIG. 1B is the molecular structure of cytosine (C); FIG. 1C is the molecular structure of guanine (G); and FIG. 1D is the molecular structure of thymine (T).

FIG. 2 is a graphical representation of Raman spectra for nucleotides A, C, G, T.

FIG. 3A is an example DNA oligo having genetic or biological nucleotides A, C, G, T; FIG. 3B is an example oligo including isotope-modified nucleotides in the leading strand; FIG. 3C is an example oligo including isotope-modified nucleotides in the lagging strand; and FIG. 3D is an example oligo including isotope-modified nucleotides in the leading strand and the lagging strand.

FIG. 4 is an example DNA oligo having isotope-modified nucleotides.

FIG. 5 is a schematic diagram of a Raman sensor set-up.

FIG. 6 is another schematic diagram of a Raman sensor set-up.

### DETAILED DESCRIPTION

As indicated above, this disclosure provides isotope-modified nucleotides for DNA data storage, the nucleotides being at least one of adenine (A), thymine (T), cytosine (C), guanine (G) and having at least one isotope of at least one of hydrogen (H), carbon (C), nitrogen (N) or oxygen (O). It is noted that although the term “nucleotide” is used herein throughout, it is actually the nucleotide base (i.e., the adenine (A), thymine (T), cytosine (C), guanine (G)) that includes the at least one isotope. A nucleotide base attached to a sugar molecule (e.g., ribose) is a nucleoside, which when attached to a phosphate forms a nucleotide.

The methodology described herein is also applicable to RNA data storage, with uracil (U) used in place of thymine (T). “Synthetic” nucleotides, which are not found in a natural A, C, G, T nucleotide set, can also be used. Synthetic nucleotides can have different atomic species (e.g., fluorine, chlorine, bromine, mercury, or sulfur) or exclude atomic species (e.g., carbon, nitrogen, oxygen, or hydrogen) from the typical naturally occurring biological nucleotides. Other molecules, in addition to nucleotides, could be modified with one or more isotopes and additionally or alternately used in place of the nucleotides; for example, the methodology described herein can be applicable to polymers and other large molecules (e.g., hexane, heptane octane, pentane, etc.).

The nucleotides or molecules, and thus the data they encode, can be read, e.g., by Surface-Enhanced Raman Spectroscopy (SERS). SERS is able to differentiate between molecules, including differentiate between molecules with different isotope concentrations. This isotope differentiation allows the same chemical compound (e.g., molecule) to represent multiple unique states.

By using isotope-modified nucleotides for DNA data storage, data density can be greatly increased due to the additional spectral signatures present beyond the traditional four signatures present in the four natural nucleotides. Overlapping spectral signatures due to molecular symmetry are expected to be detectable as sensing technology continues to evolve. In essence, the more sensitive the spectroscopic technique, the higher the potential data storage. When all possible states are resolvable with sensing technology, greater than 1 bit per atom can be realized using DNA or other suitable molecules.

Additionally, by using isotope-modified nucleotides for DNA data storage, the data is tamperproof from any reading system that makes chemical copies of the nucleotides as part of the reading process. Sensing techniques that detect isotopes (e.g., spectroscopy) will still require additional information to determine which isotope spectroscopic shifts represent data and which ones represent natural or intentionally introduced background noise.

Still further, by using isotope-modified nucleotides for DNA data storage, a limited lifetime for the data can be designed by utilizing decaying isotopes, e.g., to provide data security in niche applications.

In the following description, reference is made to the accompanying drawing that forms a part hereof and in which is shown by way of illustration at least one specific implementation. The following description provides additional specific implementations. It is to be understood that other implementations are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

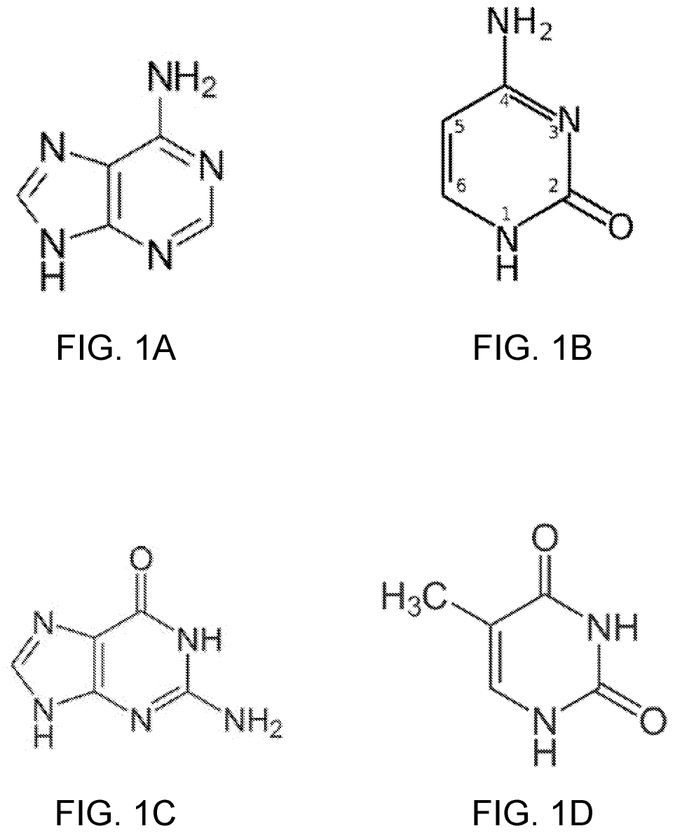

FIGS. 1A, 1B, 1C and 1D show molecular structures of the four natural biologic nucleotides that make-up DNA, adenine (A), cytosine (C), guanine (G), and thymine (T), respectively. A strand of DNA can be a carrier for data, by assigning a bit pattern to each nucleotide. Traditional biological DNA data storage is limited to four states per natural nucleotide. A data storage bit is typically represented by one nucleotide on one strand of the DNA double-helix strand, the other strand having the complementary nucleotide which offers redundancy but not extra data capacity. For example, binary bits can be arbitrarily assigned to the nucleotides as follows: A=00, G=01, C=10, and T=11. Thus, with this example, if the binary data 0000111100011110 is desired, an oligo (a portion of a DNA strand) having nucleotides in the order AATTAGTC is needed. Such an oligo can be formed by any suitable method to obtain the desired nucleotide sequence. Once the oligo is formed, it can be sequenced or “read” by any suitable method that can identify the nucleotides and convert the nucleotides identification to data bits.

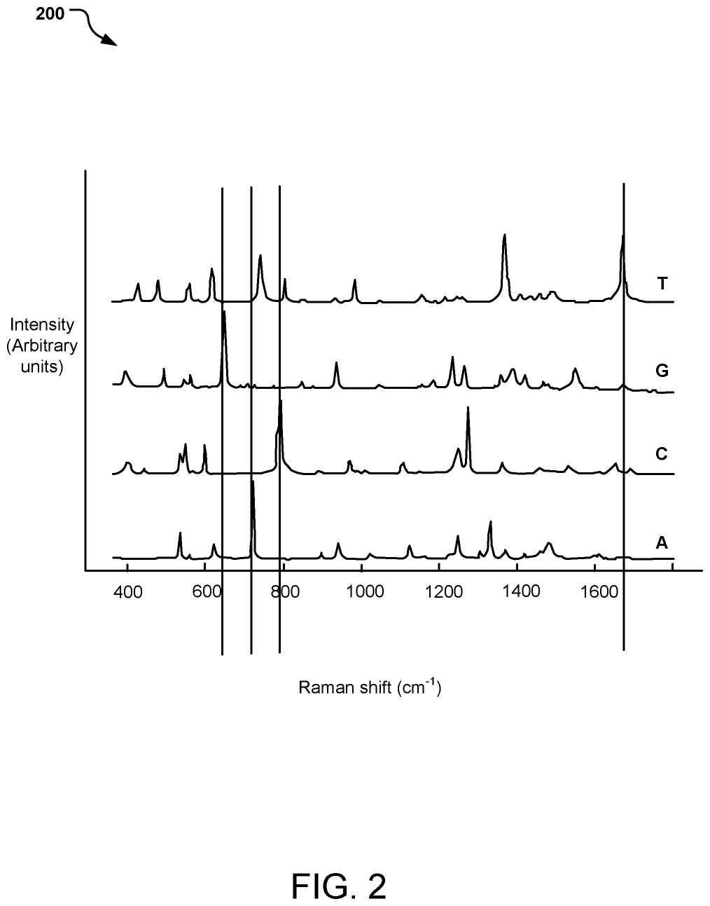

Surface Enhanced Raman Spectroscopy (SERS) is an ultrasensitive optical detection method that can be used to identify nucleotides based on their unique Raman scattering spectra. Each of the four nucleotides (adenine (A), cytosine (C), guanine (G), and thymine (T)) emits Raman-scattered photons with unique frequencies when excited by a laser. FIG. 2 shows a graph 200 of the Raman spectra of adenine (A), cytosine (C), guanine (G), and thymine (T) at an excitation wavelength of 514.5 nm. Example peaks that may be used for nucleotide identification are identified in FIG. 2: 721 cm⁻¹ for A, 776 cm⁻¹ for C, 643 cm⁻¹ for G, and 1680 cm⁻¹ for T. Using SERS, a strand of DNA can be sequenced and thus the data identified.

With the four natural biologic or genetic nucleotides, there are four states per bit (nucleotide) position. These natural nucleotides are a base 4 (quaternary) number system compared with the more commonly used base 2 (binary), base 10 (decimal), and base 16 (hexadecimal) number systems. The number of bit states (and therefore the base of the number system) can be increased by utilizing at least one isotope in a nucleotide. For example, with the addition of two isotope-modified nucleotides, the number of nucleotide states increases from four to six. By increasing the number of isotopes and where those isotopes are located in a nucleotide, the number of bit states represented by a nucleotide can be increased exponentially.

A natural nucleotide can have one of four states per position. These four states are the equivalent of 2 binary bits (4=2²). Each natural nucleotide position can therefore carry two binary bits. However, as will be shown with isotope encoding, each correlated nucleotide pair can have >2³¹ states (base 2³¹ number system) representing >15 times increase in storage density binary bits per unit volume, where the nucleotide volume is essentially constant versus the data density.

The number of states each DNA nucleotide can have is dependent on the resolution capability of the reading (e.g., spectroscopic) technique used. Higher spectroscopic resolution will support detection of smaller spectroscopic shifts which directly affects the number and position of isotopes that can be used to provide additional states for a given nucleotide. Greater spectroscopic sensitivity allows for greater number of isotopes per nucleotide, and thus greater number of states and increased data storage.

In adenine, seen in FIG. 1A, there are 5 atoms each of carbon, hydrogen, and nitrogen. If one isotope for each carbon, nitrogen and hydrogen is used (assuming there are only two possible isotopes for each of these three atomic species), there are (2⁵)(2⁵)(2⁵)=(2⁵)³=2¹⁵=32,768 unique states. This number of states is possible because the three atomic species are independent variables; e.g., the carbon atom isotope and where that carbon atom is located is not dependent on any of the other carbon, hydrogen, or nitrogen isotopes. Each grouping of (2⁵) represents one of the atomic species (C, H, or N), where 2 is the number of isotopes and 5 is the number of atoms of the atomic species in adenine. The same situation applies to all of the other atoms and atomic species in the molecule.

Referring to FIG. 1B, cytosine has one oxygen atom and thus only one possible location for an oxygen isotope. By switching this oxygen atom with one of its isotopes, there are 2¹ or 2 possible states —one state with the isotope and one state without the isotope. However, oxygen has three stable isotopes, thus there are 3¹=3 states for the nucleotide—one state with each of the isotopes O¹⁶, O¹⁷ and O¹⁸. Cytosine also has three possible locations for a nitrogen isotope, four possible locations for a carbon isotope, and five possible locations for a hydrogen isotope.

Guanine, of FIG. 1C, has one oxygen atom thus only one possible location for an oxygen isotope, five possible locations for a nitrogen isotope, five possible locations for a carbon isotope, and five possible locations for a hydrogen isotope.

Thymine, of FIG. 1D, has two oxygen atoms and two possible locations for an oxygen isotope, two possible locations for a nitrogen isotope, five possible locations for a carbon isotope, and six possible locations for a hydrogen isotope.

The number of states is an exponential relationship between the number of possible isotopes being used and the number of possible locations the isotope can be located at in the molecule. There are multiple stable and decay prone isotopes that can be used to increase the number of detectable states for a given nucleotide. For example, carbon (C) has isotopes C¹², C¹³ and radioactive C¹⁴; hydrogen has H¹ (protium), H² (deuterium) and radioactive H³ (tritium); nitrogen (N) has N¹⁴ and N¹⁵; oxygen (O) has O¹⁶, O¹⁷ and O¹⁸. Other isotopes of C, H, N and O are known but are less practical due to the isotope decay times.

As seen in FIG. 2, each of the four genetic or biological nucleotides, A, C, G, and T, emits Raman-scattered photons with unique frequencies when excited by a laser. These emitted frequencies are slightly shifted when there is a mass change, such as when an atom is replaced by one of its isotopes. When an atom in a molecule is replaced by an isotope of larger mass, mass interactions in the molecule shift the vibrational energy levels of the molecule, which can be sensed with SERS. These shifts with isotope replacement can be used to increase the number of possible states of a DNA nucleotide with no significant chemical property changes to the nucleotide.

The ability to differentiate between isotopes is dependent on the given isotope's frequency shift of the Raman-scattered photons, the location of the isotope in the molecule, and the sensitivity of the Raman spectrometer. Raman Spectroscopy including SERS is just one of the spectroscopic techniques that can be used to identify different atomic isotopes; other spectroscopic techniques (e.g., X-ray spectroscopy) can also be used. The SERS implementation described here is representative of the other spectroscopic implementations (ultra-violet, x-ray, gamma ray). Higher spectroscopic sensitivity (usually associated with higher frequencies) will yield improved state detection of overlapping frequency shifts due to molecular symmetry. This will allow for increasing data density, improving copy protection, and improving self-erasing characteristics as detector sensitivity continues to improve over time.

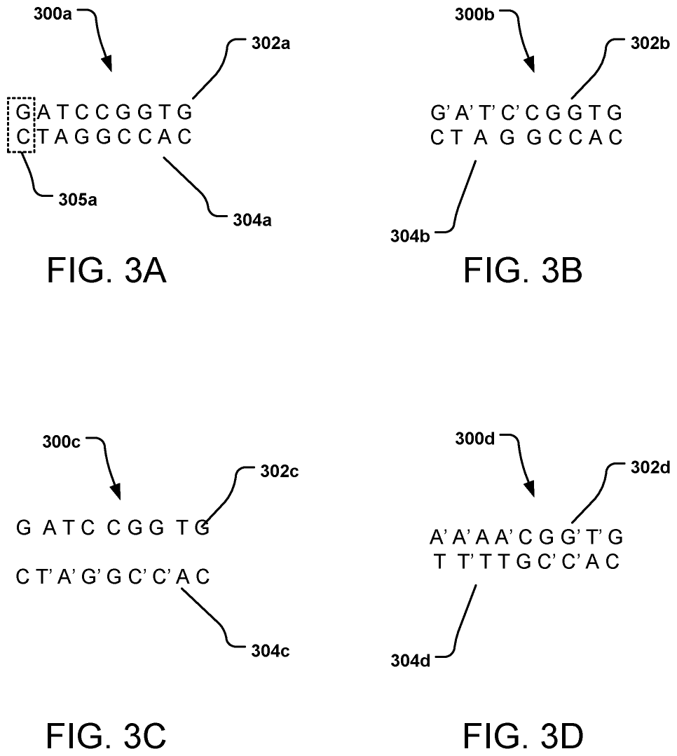

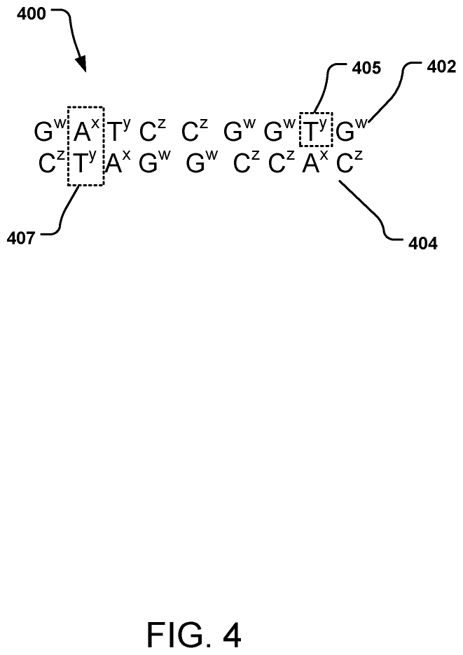

FIGS. 3A through 3D and FIG. 4 show how the locations of one to many isotopes in one or more nucleotides drastically increases the available storage density and capacity of a DNA strand. In each of these figures, the top strand is the leading strand and the bottom strand is the lagging strand, having nucleotides complementary to the leading strand.

The lagging strand nucleotide is always chemically fixed in relation to the leading strand nucleotide. In the absence of synthetic nucleotides, for DNA, guanine (G) only pairs with cytosine (C), and adenine (A) only pairs with thymine (T). As such, although the lagging strand is different it is generally redundant for data storage purposes as shown in FIG. 3A. Although the lagging strand may provide for long term chemical stability and integrity of both strands, the total information stored is just what can be stored on one strand.

However, as shown in FIG. 3C, isotope-modified nucleotides in the lagging strand can store a different data set than the leading strand while still remaining chemically bound to the leading strand. Any isotope-modified or biological adenine will pair with any isotope-modified or biological thymine, and any isotope-modified or biological guanine will pair with any isotope-modified or biological cytosine. This has the effect of increasing the information stored on a double DNA strand. The increase will vary depending on the nucleotide, as each nucleotide supports a different number of independent and non-overlapped states.

FIG. 3A illustrates an example DNA oligo 300a having nine genetic or biological nucleotide pairs arranged as a top or leading strand 302a and a bottom or lagging strand 304a. Each pair is organized vertically, and in FIG. 3A, a box 305a delineates the pair in (arbitrarily defined) position 0, with subsequent pairs representing subsequent positions 1-8. The top or leading strand 302a has nucleotides GATCCGGTG. The lagging strand 304a has the complementary nucleotides CTAGGCCAC, which offers redundancy but not extra data capacity. Using the example from above with arbitrarily assigned bit values A=00, G=01, C=10, andT=11, the leading strand 302a encodes the binary data 010011101001011101 and the lagging strand 304a encodes the binary data 101100010110100010. Although the lagging strand 304a has a different data pattern, the lagging strand data pattern will always be fixed in relation to the leading strand 302a and therefore does not store additional data.

The total possible states for any position (e.g., the position identified by the box 305a) of the leading strand 302a is four (i.e., A, C, G, T). Each natural genetic or biological nucleotide position supports only four possible states.

FIG. 3B illustrates how multiple isotopes can be used to increase the number of distinct states that can be recognized per bit (nucleotide) versus the biological nucleotides in FIG. 3A (assuming the isotopes' spectroscopic shifts can be resolved as unique with a suitable measurement technique (SERS, x-ray, gamma ray, etc.)). FIG. 3B shows an example DNA oligo 300b having nine nucleotide pairs, wherein four of the nucleotides include one isotope, the isotope-modified nucleotides being represented with a “prime.” Particularly, the top or leading strand 302b has nucleotides G′A′T′C′CGGTG. The isotope-modified nucleotides are spectroscopically different from the related biological nucleotide due to the presence of the isotope. There are eight total possible states for any position of the leading strand 302b (i.e., A, A′, C, C′, G, G′, T, T′). As in FIG. 3A, the bottom or lagging strand 304b has the complementary biological nucleotides CTAGGCCAC, none of which are isotope-modified, so that the total possible states for any position of the lagging strand 304b is still four (i.e., A, C, G, T). Because of the difference between the isotopes of the leading strand 302b and the lagging strand 304b, the strands 302b, 304b carry different data. Even though the leading strand 302b supports eight states, the lagging strand 304b is still dependent on the leading strand. While G or G′ on the leading strand is possible, only C is possible on the paired lagging strand; thus, the total number of states per position remains at eight.

FIG. 3C shows an example DNA oligo 300c having nine nucleotide pairs, with various nucleotides in the lagging strand being isotope-modified. Similar to the oligo 300b in FIG. 3B, the leading strand 302c and the lagging strand 304c carry different data, although the lagging strand 304c is still dependent on the leading strand. For example, while only G on the leading strand is possible, both C and C′ are possible on the lagging strand paired to the G; thus, the total number of states per position still remains at eight.

FIG. 3D shows an example DNA oligo 300d also having nine nucleotide pairs, with various nucleotides in both the leading strand 302d and the lagging strand 304d being isotope-modified.

The examples of FIGS. 3B, 3C and 3D have assumed that only one isotope, at one location, is present in the isotope-modified nucleotide. However, multiple isotopes of a single atom and multiple isotopes at different locations can be used to increase the amount of data present, subject to the resolution of the spectroscopic technique being used. There will be some overlap of states due to molecular symmetry that will be difficult or impossible to resolve, and that will reduce the total realizable states in a physical system. However, with more sensitive equipment and techniques, it may be possible to resolve all states with future sensor designs.

Whether only one isotope or multiple, the leading strand 302 and the lagging strand 304 can be interpreted by a “reader” in one of two methods. The first method is as described above in respect to FIG. 3B and FIG. 3C (i.e., even though the leading strand 302c, 302d and the lagging strand 304c, 304d are complementary, each strand carries a unique set of information when isotopes are included). The second method is by correlating the information in the strands 302, 304, so that the relative position of the nucleotide in the leading strand 302 and the lagging strand 304 is fixed.

Correlating the strands 302, 304 increases the size of the data set that can be represented in the overall strand 300. Any one position in the strand 300 now supports sixteen states—AT, AT′, A′T, A′T′, TA, TA′, T′A, T′A′, CG, CG′, C′G, C′G′, GC, GC′, G′C, G′C′. Synchronizing data from both the leading strand 302 and lagging strand 304 has a multiplicative effect on states represented, compared to an additive effect when data is only read from one strand (e.g., the leading strand). A strand tagging method can be used can be used to ensure data can be synchronized.

FIG. 4 shows a DNA oligo 400 also having nine nucleotide pairs, with a leading strand 402 and a lagging strand 404. FIG. 4 shows schematically how each nucleotide in both the leading strand 402 and the lagging strand 404 can have an exponential number of different isotope-modified states for the example double stand, where the superscripts w, x, y, and z represent the total number of states that the isotope modified nucleotides G, A, T, and C can have, respectively. FIG. 4 also shows an uncorrelated nucleotide denoted by a box 405 and a correlated pair denoted by box 407.

For a non-correlated strand, the two strands 402, 404 do not need to be read simultaneously or even together, and each position (e.g., a nucleotide in the position of the box 405) in the leading strand 402 or in the lagging strand 404 can support a different number of states depending on the nucleotide present. The data present in the position of the box 405 shows thymine supporting “y” unique states. The number of unique states (e.g., “y”) is dependent on the number and atomic species of the isotopes in the (e.g., thymine) molecule. Other nucleotides will have different numbers of unique states, as has been discussed above. The number of unique states is not dependent on the nucleotide with which it is paired.

For a correlated strand, the relative position between the leading and lagging strands 402, 404 is relevant and must be known at all times, as the nucleotides in the two strands are paired; FIG. 4 shows a correlated pair denoted within the box 407. In the box 407, nucleotides A and T are “paired,” so that both strands are read simultaneously; because of this, the number of data states represented is the multiplied product of x and y (i.e., x*y), rather than x (the number of states of A) or y (the number of states of T), nor x+y (the number of states for the pair if not correlated).

Although the strands 402, 404 are correlated, it is not necessary to read both strands simultaneously, rather each strand can be read individually as long as the position (e.g., any one of positions 0-8) of the leading strand 402 and lagging strand 404 nucleotides are known. The strands 402, 404 can be tagged or otherwise have the position(s) identified or indexed, particularly if the strands 402, 404 are processed separately.

Returning to FIGS. 1A through 1D, the molecular structures for the biologic nucleotides of DNA are illustrated and have the formulas: adenine-C₅H₅N₅, thymine-C₅H₆N₂O₂, cytosine-C₄H₅N₃O, and guanine-C₅H₅N₅O. Hydrogen (H), carbon (C), nitrogen (N) and oxygen (O) have (at least) the following stable isotopes, respectively: H¹, H², C¹², C¹³, N¹⁴, N¹⁵, O¹⁶, O¹⁷, and O¹⁸. Any or all of these isotopes can be used in any appropriate location in each or any of the nucleotides.

Each nucleotide supports a different number of isotopic states due to the individual atomic makeup of the nucleotide. The AT paring supports more individual states (approximately double) than the CG paring, before accounting for symmetry. In some implementations, using the AT pairing exclusively can be done to maximize the data stored, as long as the DNA double strand remains stable with just one nucleotide paring present.

By using the formula Num_isotopes^{Num_atoms}, the total independent states for a nucleotide, taking into account all possible isotope locations for each isotope, can be calculated. Thus, each isotope-modified nucleotide has a number of possible states defined by:

number of possible states=(a^{Na})*(b^{Nb})*(c^{Nc})* . . . (z^{Nz}) (I)

where:

a, b, c . . . z is the number of isotopes available for a given atom, and

Na, Nb, Nc . . . Nz is the number of atoms of that identified element represented by isotopes (i.e., a, b, c, a . . . z) in the nucleotide.

Returning to FIG. 4, for thymine, the number of possible states (represented by the superscript “y” in FIG. 4) is 2⁵*2⁶*2²*3²=73,728, based on: 2 carbon isotopes for 5 carbon atoms, 2 hydrogen isotopes for 6 hydrogen atoms, 2 nitrogen isotopes for 2 nitrogen atoms, 3 oxygen isotopes for 2 oxygen atoms. Similarly, the number of states supported by the other natural nucleotides are: adenine “x”=2⁵*2⁵*2⁵=32,768; guanine “w”=2⁵*2⁵*2⁵*3¹=98,304; and cytosine “z”=2⁴*2⁵*2³*3¹=12,288.

The number of states available to a correlated position in the strand (e.g., denoted by the box 407) is much greater than to a non-correlated position (e.g., denoted by the box 405). Each non-correlated position in the strand can represent 218,088 possible (different) isotope-modified nucleotide states (i.e., 73,728+32,768+98,304+12,288=218,088), whereas a correlated position in the strand has significantly more possible (different) isotope-modified nucleotide states, >2³¹ or >2³⁰ (i.e., 32,768*73,728=2,415,919,104 for an AT pair or 12,288*98,304=1,207,959,552 for a CG pair).

If both the leading and lagging strands are processed independently (i.e., they are not correlated), the AT or CG pair may make up the entire double strand, provided the DNA can remain stable in that configuration. An example of this is shown in the first four positions of FIG. 3D. As an example, the leading strand 302d could be all adenine (A), and each adenine position would represent 32,768 (2¹⁵) states. The lagging strand 304d would thus be all thymine (T) with each position representing 73,728 (>2¹⁶) states. This would be similar if the oligo were only composed of the CG pair.

For non-correlated reading or decoding, each position of the AT pair would support 32,768+73,728 states and each CG pair would support 12,288+98,304 states. However, if both the leading and lagging strands 302d, 304d were correlated while encoding and decoding (processed dependently), as shown by the pair in the box 407 in FIG. 4, then the data stored between the leading and lagging nucleotide pair is not the summation of the two paired nucleotides, but the multiplication of each paired nucleotides' possible states. Thus, the AT pair supports 73,728*32,768=2,415,919,104 states per position, which is >2³¹ states per position.

With 2³¹ total possible states represented by 30 atoms from the AT pair, there is >1 binary bit per atom storage density possible in the pair. The GC pair support 1,207,959,552 states (>2³⁰) per position, essentially half of the AT pair.

With correlated decoding of the two strands, the order of the leading strand to the lagging strand has an effect; i.e., AT is uniquely different from TA and CG is uniquely different from GC, providing different data and a different number of possible states. The total possible states for a single position of a nucleotide pair is AT+TA+CG+GC, which is 7,247,757,312 possible states (>2³²). If a nucleotide with a long half-life (e.g., carbon14) is included, it will add long term data decay, and will increase the possible states to >2³⁸ (1.3 binary bits per atom).

With today's technology, many of the state combinations may not be resolvable, for example, with Raman scattering or surface enhanced Raman scattering (SERS). However future techniques (e.g., x-ray spectroscopy) are expected to be able to resolve more states. Other spectrographic techniques may also be useable. As the ability to resolve more states due to increased sensitivity improves, so will data storage density. The higher the resolution of the sensing technique, the greater the ability to differentiate symmetrical combinations and the greater the amount of data that can be stored on a given isotope-modified nucleotide, approaching the theoretical states calculated above. Isotope-modified nucleotides for DNA data storage have the potential to exceed >1 bit state of storage per atom as the sensitivity of the detector improves over time.

Isotope modified nucleotides have a unique property which is a variable number base system for storing data. The number base is defined by the number of states that are encoded, and the number of possible states is determined by which isotope combinations are used in the encoding. This state information is created and utilized as needed by the data encoder.

Not only does utilizing isotope-modified nucleotides drastically increase the data storage density on a DNA strand, copying of the DNA strand is prohibitive, which adds a level of security to the data.

In some methodologies, when data is read from DNA, multiple copies of the DNA strand are created. These copies are processed in parallel and the read data is combined to obtain a full data set from the original strand. This technique is conventionally used because reading an entire length of a strand of DNA can take a long time with standard techniques, whereas processing multiple copies at the same time has the effect of increasing the speed of reading the DNA nucleotide values. SERS, as discussed in respect to FIG. 2, does not require multiple strand copies to read the data.

As indicated directly above, copies of the DNA strand are commonly made, e.g., to hasten reading. However, a chemical process cannot copy the isotope information in an isotope-modified strand, as disclosed herein, as all isotopes of a single element, and hence the resulting nucleotide, are chemically identical. In such a manner, although a chemical copy can be made, the copy will not include the isotope information and therefore that copy is not a true duplicate, thus providing a mode of copy protection, because the data is protected from common chemical copying processes. In this copy protection methodology, the unintended reader, without additional information on how nucleotide encoding is being used (e.g., which isotopes, where in the nucleotide, which nucleotides, number of isotopes per nucleotide, etc.) or whether it is being used, will not know data was lost with the chemical copy, and thus will be unable to know, much less effectively decode, the data. Thus, by using isotope-modified DNA for data storage, the data is protected from common chemical copying and reading.

Another reading process for DNA data uses spectroscopic techniques, e.g., Raman spectroscopy. However, without prior knowledge as to which nucleotides should have isotopic shifts in the spectroscopy, the unintended reader will not know if a measured spectroscopic shift is due to an expected isotope and hence part of the data or if it is background noise. Additionally, the unintended reader may overlook the encoded data completely if the reading technique is not sensitive enough to recognize the small shifts in the isotope spectroscopic response. Again, by utilizing isotope-modified DNA for data storage, the data is protected from common spectroscopic analysis. The data is also protected from the unintended reader by the number base used in the encoding. Only the encoder and the intended reader know the number base being used. Any number base can be chosen between 2¹ and 2³² to encode the data when using the techniques described.

It is noted that to have a viable spectroscopic copy protection, the concentration of the isotopes in the DNA should be taken into account. Too much variation from natural spectral levels can suggest to the unintended reader the presence of isotopic-modification in the nucleotides, although the unintended reader would nevertheless need to determine how the nucleotide encoding is being used (e.g., which isotopes, where in the nucleotide, which nucleotides, number of isotopes per nucleotide, etc.).

Higher levels of less common isotopes can be used to flood the spectroscopic response, thus hiding the true data present in only pre-defined specific shifts. Flooding the signal, in this manner, complicates attempts to determine which isotope locations represent the encoded data.

Offsetting correlated strands is another technique to protect isotope encoded data from unintended viewing. When two strands (e.g., strands 402, 404 of FIG. 4) are correlated, their relative positions need to be known; it is not necessary that the strand correlation be adjacent as shown in FIG. 4 by the box 407. The correlation between the leading and lagging strands can be adjusted as needed, e.g., shifted one or more nucleotides. As an example, referring to FIG. 4, the encoding process could define having G in position 0 of the leading strand 402 (the first of the correlated data pair) and T in position 1 of the lagging strand 404 (the second of the correlated data pair). Thus, although G and T are not complementary nucleotides and they are not in the same position, this G and T are correlated for data encoding. Any pattern can be used when correlating one position of the leading strand to a position of the lagging strand to mask the actual data from the unintended reader.

As indicated above, not only does utilizing isotope-modified nucleotides drastically increase the data storage density on a DNA strand and inhibit copying and identification of the DNA strand, the data can be designed with a limited lifetime, or, designed with a “self-destruct” mechanism. A limited data life can be implemented using short-lived isotopes in an isotope-modified nucleotide.

When an isotope decays, the spectroscopic information changes to a new state and the value no longer reflects the original recorded data. Depending on the resulting decayed atom, the molecule (nucleotide) may also become unstable and break up. Examples of decay-prone isotopes that can be used to encode data in a nucleotide include tritium (12.32 year half-life) and phosphorous 33 (25 day half-life). Tritium (H³) is a particularly good candidate isotope for self-erasing or limited life data. The natural nucleotides contain about 30% hydrogen, and tritium can break the nucleotide bonds when it converts to Helium3 (He³). Once the nucleotide bonds are broken, order is lost and the data is permanently scrambled. When designing a limited life for an isotope-modified nucleotide, the isotope percentage should be sufficiently high that the decayed state cannot be overturned with error correction techniques.

To read the DNA strand having at least one isotope-modified nucleotide, numerous technologies may be used. Raman spectroscopy is one suitable technology.

A Raman sensor or device can be used that has a Raman “hot spot” channel formed by laser excitation and enhanced by resonance of focusing plasmonic (e.g., gold, silver) nanostructures. A DNA template strand is drawn or fed through the hot spot channel. As the DNA template strand moves through the hot spot, Raman spectra for the individual nucleotides and isotope-modified nucleotides are measured.

In some implementations, rather than measuring each nucleotide individually, the Raman spectra for a first group of nucleotides present in the hot spot channel is measured at a first point in time, and the Raman spectra for a second group of nucleotides present in the hot spot channel is measured at a second point in time subsequent to the first point in time. The two Raman spectra are compared to determine what nucleotide(s) left the hot spot and what nucleotide(s) entered the hot spot.

In some implementations, the device includes a DNA polymerase, which replicates the template strand being sequenced. The replication action by the polymerase pulls the template strand through the hot spot channel. In some implementations, a secondary force, e.g., an electric force or voltage differential, is additionally or alternatively used to aid the passage of the strand through the hot spot channel between the nanostructures.

The sensor can be provided as a microfluidic lab-on-a-chip system, or, “on chip.”

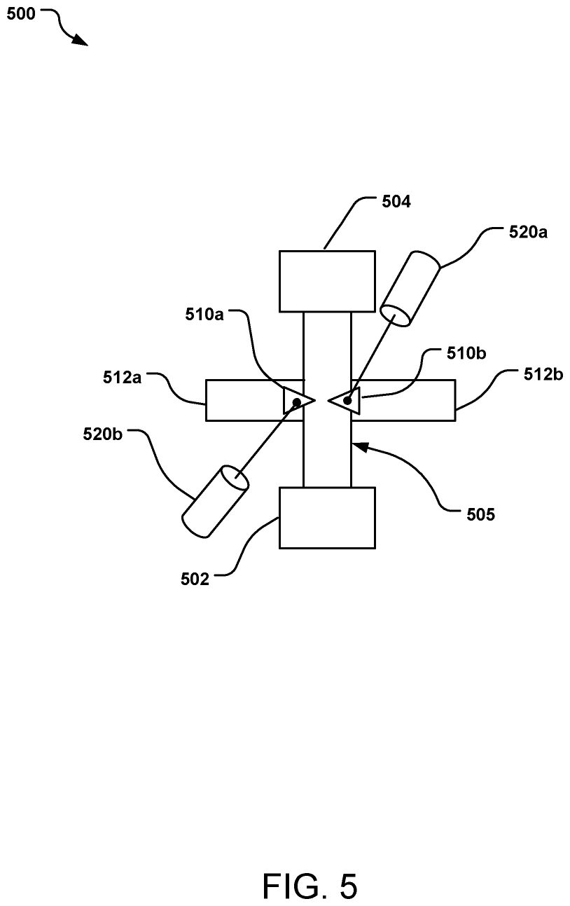

FIG. 5 generally illustrates a SERS (surface enhanced Raman scattering) sensor 500 for sequencing a DNA template strand. The sensor 500 has a sample loading chamber 502, a secondary or sample receiving chamber 504 and a nanochannel 505 connecting the chambers 502, 504. A pair of nanostructures 510a, 510b is located on opposites sides of the nanochannel 505, operably connected to a pair of waveguides 512a, 512b. The nanostructures 510 focus the Raman signal to a small region (e.g., 1-10 nm wide) in the nanochannel 505. The nanostructures 510 may be any of a variety of shapes, such as triangular (as in FIG. 5), lollipop, other pointed surface designs, etc. Two oppositely positioned triangular nanostructures resemble a bow tie, and two oppositely positioned lollipop nanostructures resemble a dumbbell. The nanostructures 510 may be two-dimensional or three-dimensional. Tapered or pointed nanostructures 510 are particularly useful for focusing the signal.

The nanostructures 510 are plasmonic nanostructures and may be made of gold, silver, platinum or another plasmonic material, or a combination of plasmonic and other materials.

At least one laser 520 is focused on at least one of the nanostructures 510, in the region of the nanochannel 505; FIG. 5 shows two lasers 520a, 520b, each focused on a nanostructure 510. In some implementations, multiple lasers 520 are used for each pair of nanostructures; thus, for two pairs (four) nanostructures, at least four lasers are used.

The laser(s) 520 are directed at the nanostructures 510 and/or the gap between them, to generate plasmons across the nanostructures 510 and create a Raman hot spot in the nanochannel 505. The one or more waveguides 512 may be used to direct the laser beam(s) to the nanostructures 510. The laser(s) 520 may be, individually, e.g., a solid state laser, a gas (e.g., xenon) laser, a liquid laser, etc., or any similar light source operating at, e.g., 600 nm, 800 nm, 1064 nm wavelengths. Multiple lasers 520 may be positioned parallel to or perpendicular to the nanostructures and may be on the same plane or a separate plane.

The resulting Raman photons or light scattered by the nucleotides (hence, the Raman spectra) are measured and the nucleotides identified. Stokes scattered photons, Anti-Stokes scattered photons, or both may be used for nucleotide identification. The Raman scattered photons may be collected and/or focused by mirrors or lenses to facilitate identification of the nucleotides, or the scattered light may be collected by a waveguide. Light may be detected and quantified by a photomultiplier tube, photodiode array, charge-coupled device, electron multiplied charge-coupled device, etc. The resulting Raman-scattered photons may be filtered such that only photons of specific frequencies are detected. In some implementations, optical resonator(s) may be present to increase the signal from the detected photons.

In use of the sensor 500, a DNA template strand having one or more isotope-modified nucleotides is drawn or fed from the sample loading chamber 502 through the nanochannel 505 through the hot spot formed by the nanostructures 510 and the laser(s) 520. The laser(s) 520, focused on the nanostructures 510, enhance the Raman spectra or resonance obtained from the scattered photons, allowing each individual nucleotide to be identified by its Raman spectra.

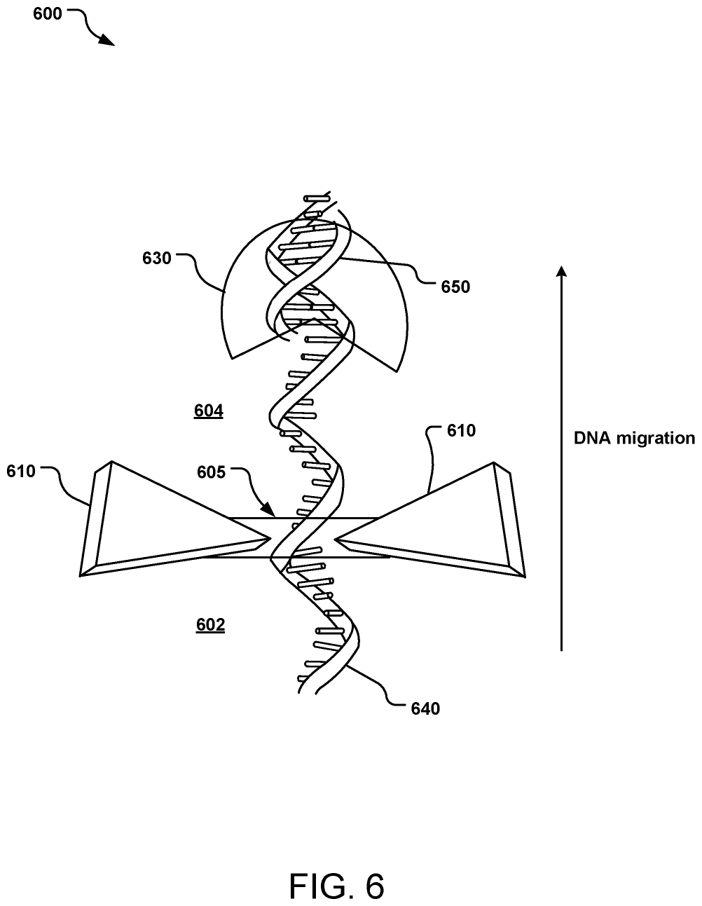

In FIG. 6, a SERS sensor 600 is schematically illustrated, almost in a cartoon manner. Only certain features of the sensor 600 are shown in FIG. 6; it is to be understood that the sensor 600 includes other features (e.g., laser(s)) as described in relation to FIG. 5.

The sensor 600 has a sample loading chamber 602, a secondary chamber 604, and a nanochannel hot spot 605 therebetween. This nanochannel hot spot 605 is generated by laser excitation and enhanced by resonance of metallic (e.g., gold) nanostructures 610. The sample loading chamber 602 is upstream of the nanochannel hot spot 605 and the secondary chamber 604 is downstream of the nanochannel hot spot 605.

A DNA polymerase 630 (illustrated as a Pac Man™ type shape) replicates a DNA template strand 640 to be sequenced, the strand having at least one isotope-modified nucleotide; the replication process, however, is not able to replicate the isotope information, as discussed above. The replicated complementary strand 650 is shown proximate the DNA polymerase 630. The action of replicating the template strand 640, by the DNA polymerase 630, applies a tension or force on the strand 640 and pulls the strand through the Raman nanochannel hot spot 605. Each of the nucleotides of the template strand 640 generates a unique Raman signal depending on its identity as it passes through the nanochannel hot spot 605.

The nucleotides present in the nanochannel hot spot emit Raman-scattered photons, which can then be filtered and detected. Each of the nucleotides A, C, G, T emits Raman photons of specific frequencies (see, FIG. 2), and any isotope in those nucleotides affects the emitted frequency. The amplitude of the signal intensity at a selected frequency can be used to identify the nucleotide (e.g., isotope-modified nucleotide) and thus the data it encodes.

Various additional and alternate implementations are also contemplated.

In some implementations, the DNA template strand is a linear single strand (as shown, e.g., in FIG. 6 as template strand 640), whereas in other implementations the strand entering the hot spot is a double strand. A double strand is sequenced in the same manner as a single strand.

In other implementations, a DNA exonuclease, an RNA polymerase or exonuclease may be used in place of a DNA polymerase or DNA exonuclease, in order to sequence RNA or DNA. Alternately, an electric current or voltage differential may be used to pull the strand through the hot spot(s) or aid in the pulling. Other sources of electrophoresis may additionally or alternatively be used, as well as another source of force, e.g., electromechanical.

In summary, described herein is the use of isotope-modified nucleotides and other molecules for encoding data thereon. Any or all of the H, C, N and O molecules can be replaced with an isotope, thus modifying the nucleotide. Each modified nucleotide will produce a different Raman scattering spectra. Thus, the more and/or different isotopes in the nucleotide, the more nucleotide signatures, and the more nucleotide signatures, the grater the increase in the data density available in the DNA strand. Rather than each nucleotide having only one data state available and encoding 2 bits (e.g., 00, or 01, or 10, or 11), the number of possible states is a function of the number of isotope-replaceable-atoms and the number of available isotopes. As shown above, thymine theoretically has 73,728 data states, adenine theoretically has 32,768 data states, guanine theoretically has 98,304 data states, and cytosine theoretically has 12,288 data states. Thus, each modified nucleotide can encode significantly more bits. Additionally, if the processing of the two strands is correlated (where position matters), the data store in any nucleotide pair position exceeds 2³² states (32 bits).

The above specification and examples provide a complete description of the structure and use of exemplary implementations of the invention. The above description provides specific implementations. It is to be understood that other implementations are contemplated and may be made without departing from the scope or spirit of the present disclosure. The above detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the disclosure or the recited claims.

What is claimed is:

1. A method of storing data on a molecule, the method comprising:
providing a first molecule having a molecular structure with at least one isotope within the structure and a second molecule having the molecular structure without an isotope; and
assigning a bit pattern to the first molecule that is different than a bit pattern assigned to the second molecule.

2. The method of claim 1, the method comprising:
providing a DNA strand having a first isotope-modified nucleotide comprising at least one-isotope of carbon, nitrogen, oxygen or hydrogen; and
assigning a bit pattern to the first isotope-modified nucleotide that is different than a bit pattern assigned to a non-isotope-modified nucleotide.

3. The method of claim 2, wherein both the first isotope-modified nucleotide and the non-isotope-modified nucleotide are one of adenine (A), cytosine (C), guanine (G), and thymine (T).

4. The method of claim 2, wherein providing a DNA strand having the first isotope-modified nucleotide comprises providing a DNA strand having at least one isotope-modified nucleotide modified with two isotopes.

5. The method of claim 4, wherein the two isotopes are different isotopes of the same base atom.

6. The method of claim 4, wherein the two isotopes are isotopes of two different base atoms.

7. The method of claim 2, wherein providing the DNA strand comprises providing the DNA strand having the first isotope-modified nucleotide combined with the non-isotope-modified nucleotide as a complementary pair.

8. The method of claim 2, wherein providing the DNA strand comprises providing the DNA strand having the first isotope-modified nucleotide with a first isotope in a first position, a second isotope-modified nucleotide with the first isotope in a second position different from the first position, and the non-isotope-modified nucleotide, where the first isotope-modified nucleotide with a first isotope in a first position has a first bit pattern assigned, the second isotope-modified nucleotide with the first isotope in the second position has a second bit pattern assigned different than the first bit pattern, and the non-isotope-modified nucleotide has a third bit pattern assigned different than the first bit pattern and different than the second bit pattern.

9. The method of claim 2, wherein providing the DNA strand having the first isotope-modified nucleotide comprises providing a DNA strand having the first isotope-modified nucleotide modified with a decay-prone isotope.

10. A method of reading data from a DNA data strand, the method comprising:
reading a spectral signature of a first isotope-modified nucleotide comprising at least one isotope of carbon, nitrogen, oxygen or hydrogen and determining a first bit pattern assigned to the spectral signature; and
reading a spectral signature of a non-isotope-modified nucleotide and determining a second bit pattern assigned to the spectral signature, the second bit pattern different from the first bit pattern;
both of the first isotope-modified nucleotide and the non-isotope-modified nucleotide being a same one of adenine (A), cytosine (C), guanine (G), and thymine (T).

11. The method of claim 10, further comprising:
reading a spectral signature of a second isotope-modified nucleotide comprising at least one isotope of carbon, nitrogen, oxygen or hydrogen, the second isotope-modified nucleotide different than the first isotope-modified nucleotide, and determining a third bit pattern assigned to the spectral signature, the third bit pattern different from the first bit pattern and the second bit pattern, the second isotope-modified nucleotide being paired with one of the first isotope-modified nucleotide and the non-isotope-modified nucleotide in the DNA strand.

12. The method of claim 11, wherein the second isotope-modified nucleotide paired with one of the first isotope-modified nucleotide and the non-isotope-modified nucleotide are correlated.

13. The method of claim 12, wherein the correlated pair of the second isotope-modified nucleotide and one of the first isotope-modified nucleotide and the non-isotope-modified nucleotide are offset in position in the DNA strand.

14. The method of claim 11, further comprising:
reading a spectral signature of a third isotope-modified nucleotide comprising at least one isotope of carbon, nitrogen, oxygen or hydrogen, the third isotope-modified nucleotide different than the first isotope-modified nucleotide, and determining a fourth bit pattern assigned to the spectral signature, the fourth bit pattern the same as the first bit pattern.

15. A DNA strand encoding data, the DNA strand comprising:
at least one non-isotope-modified nucleotide having a first bit pattern assigned thereto; and
at least one isotope-modified nucleotide comprising at least one isotope of one of carbon, nitrogen, oxygen or hydrogen, the isotope-modified nucleotide having a second bit pattern assigned thereto different than the first bit pattern.

16. The DNA strand of claim 15, wherein the at least one isotope-modified nucleotide and the non-isotope-modified nucleotide are independently one of natural nucleotides adenine (A), cytosine (C), guanine (G), or thymine (T), or a synthetic nucleotide comprising at least one atom that is not carbon, hydrogen, nitrogen, or oxygen.

17. The DNA strand of claim 15 comprising at least one isotope-modified nucleotide modified with two isotopes, the two isotopes are different isotopes of the same base atom.

18. The DNA strand of claim 15 comprising at least one isotope-modified nucleotide modified with two isotopes, the two isotopes are isotopes of two different base atoms.

19. The DNA strand of claim 15 comprising:
the non-isotope-modified having the first bit pattern assigned thereto;
a first isotope-modified nucleotide comprising an isotope in a first position, the first nucleotide having a second bit pattern assigned thereto different than the first bit pattern; and
a second isotope-modified nucleotide comprising the isotope in a second position different than the first position, the second nucleotide having a third bit pattern assigned thereto different than the first bit pattern and different from the second bit pattern.

20. The DNA strand of claim 15 comprising a leading strand and a lagging strand each comprising multiple nucleotides, each nucleotide having a bit pattern assigned thereto, the leading strand nucleotides and the lagging strand being non-correlated and having different sequences of bit patterns.

21. A system for data storage on a DNA strand, the system comprising:
a plurality of isotope-modified molecules, each isotope-modified molecule comprising at least one isotope, and each isotope-modified molecule having a number of possible states defined by:
number of possible states=(a^{Na})*(b^{Nb})*(c^{Nc})* . . . (z^{Nz})
where:
a, b, c . . . z is the number of isotopes available for a given atom in the molecule, and
Na, Nb, Nc . . . Nz is the number of atoms of type a, b, c, and z in the molecule,
further where each unique molecule has a unique bit pattern.

22. The system of claim 21 comprising:
a plurality of isotope-modified nucleotides, each isotope-modified nucleotide comprising at least one isotope, and each isotope-modified nucleotide having a number of possible states defined by:
number of possible states=(a^{Na})*(b^{Nb})*(c^{Nc})* . . . (z^{Nz})
where:
a, b, c . . . z is the number of isotopes available for a given atom in the nucleotide, and
Na, Nb, Nc . . . Nz is the number of atoms of type a, b, c, and z in the nucleotide,
further where each unique nucleotide has a unique bit pattern.
